(19) United States
(12) Plant Patent Application Publication
(10) Pub. No.: US 2016/0249506 P1
(43) Pub. Date: Aug. 25, 2016

(54) Raspberry plant named "Pacific Gema"

(50) ;

(71) Applicants: Ellen Thompson, Watsonville, CA (US); John Reuben Clark, Fayetteville, AR (US)

(72) Inventors: Ellen Thompson, Watsonville, CA (US); John Reuben Clark, Fayetteville, AR (US)

(73) Assignee: Pacific Berry Breeding, LLC, Salinas, CA (US)

(21) Appl. No.: 14/544,823

(22) Filed: Feb. 24, 2015

(51) Int. Cl. A01H 5/00

(52) U.S. Cl. PLT/204

(57) ABSTRACT

A new and distinct cultivar of Raspberry plant named ‘Pacific Gema’ as described and shown herein. ‘Pacific Gema’ provides balanced heavy crop loads on both primocanes and floricanes, large conic fruits that are flavorful and aromatic, high vigor and strong field tolerance to Raspberry Bushy Dwarf Virus (RBDV).

### BACKGROUND AND SUMMARY

The new raspberry cultivar designated ‘Pacific Gema’ is shown herein. Botanically recognized as Rubus idaeus L., ‘Pacific Gema’ is a primocane-fruiting raspberry resulting from a hand-pollinated cross of female parent ‘Pacific Deluxe’ (U.S. Plant Pat. No. 21,074), a previous release from the same program, and the unpatented male parent ‘414a’.

The present cultivar, ‘Pacific Gema’ offers many advantages over the existing, patented cultivar ‘Pacific Deluxe’, U.S. Plant Pat. No. 21,074, in that the root and subsequent cane vigor is significantly greater, floricane budbreak is more uniform, average fruit size is larger and more conic in shape. The increased vigor offers advantages in propagation, particularly in a nursery setting where roots are produced for sale to commercial growers. The improved floricane budbreak provides growers with a more reliable spring crop. The larger berries help growers fill baskets more quickly, leading to increased harvest efficiency.

A second example of an existing, patented cultivar is ‘Pacific Majesty’, U.S. Plant Pat. No. 23,593, another previous release from the same program. The present cultivar, ‘Pacific Gema’ has superior flavor/aroma and higher field tolerance of Raspberry Bushy Dwarf Virus (RBDV) compared with ‘Pacific Majesty’.

A third example of an existing, patented cultivar is ‘Rafzaqu’, U.S. Plant Pat. No. 19,512 (aka Himbo Top®). The present cultivar, ‘Pacific Gema’ has firmer fruit and more berries per inflorescense compared with ‘Rafzaqu’, though both are of similar height.

Thus, these characteristics help define ‘Pacific Gema’ as a new and distinct cultivar of primocane-fruiting raspberry. ‘Pacific Gema’ may be recognized by its high vigor, uniform floricane budbreak, tall canes which are architecturally laden with conic berries and which consistently taste sweet and aromatic.

### BRIEF DESCRIPTION OF THE PHOTOGRAPHS

FIG. 1 is a photograph showing the canopy of the Raspberry cultivar ‘Pacific Gema’.

FIG. 2 is a further photograph showing the canopy of the Raspberry cultivar ‘Pacific Gema’.

FIG. 3 is a close-up photograph showing ripe and unripe fruit and leaf detail on laterals of the Raspberry cultivar ‘Pacific Gema’.

FIG. 4 is a further close-up photograph showing ripe and unripe fruit and leaf detail of the Raspberry cultivar ‘Pacific Gema’.

FIG. 5 is a copy of the UPOV technical questionnaire for the Raspberry cultivar ‘Pacific Gema’.

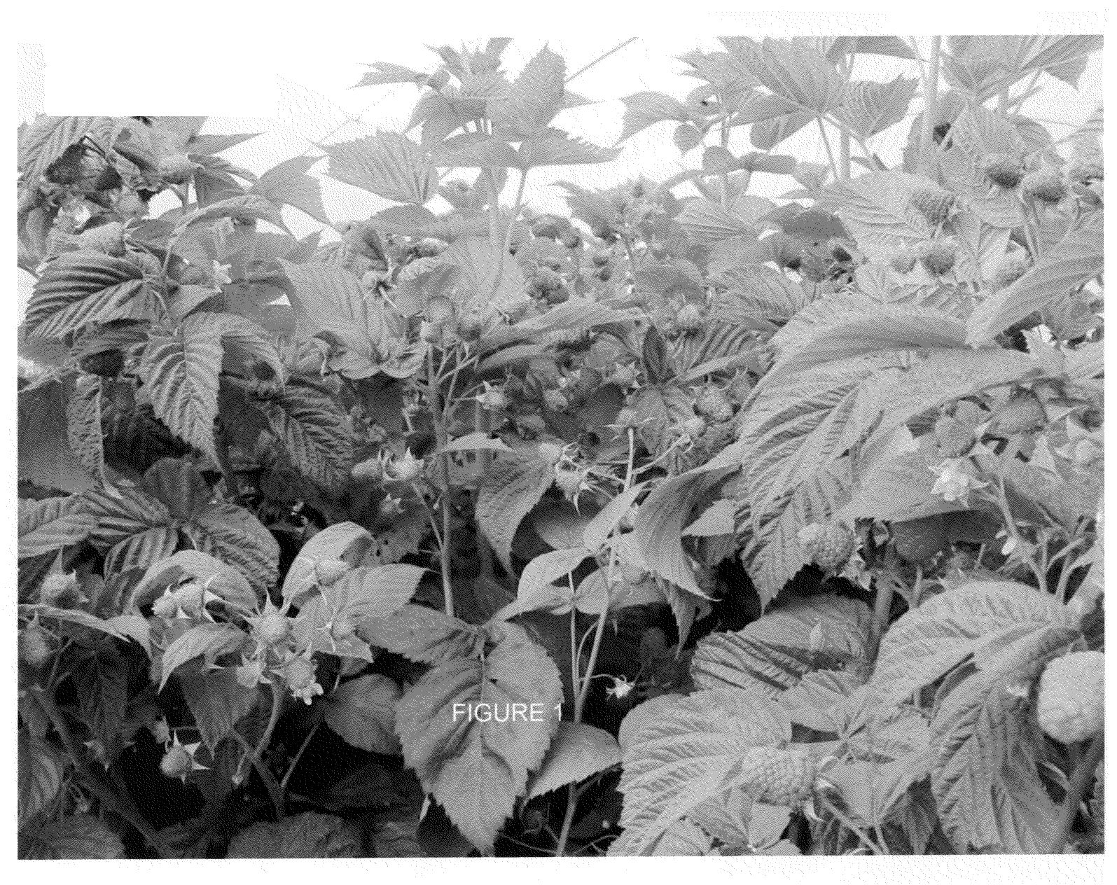

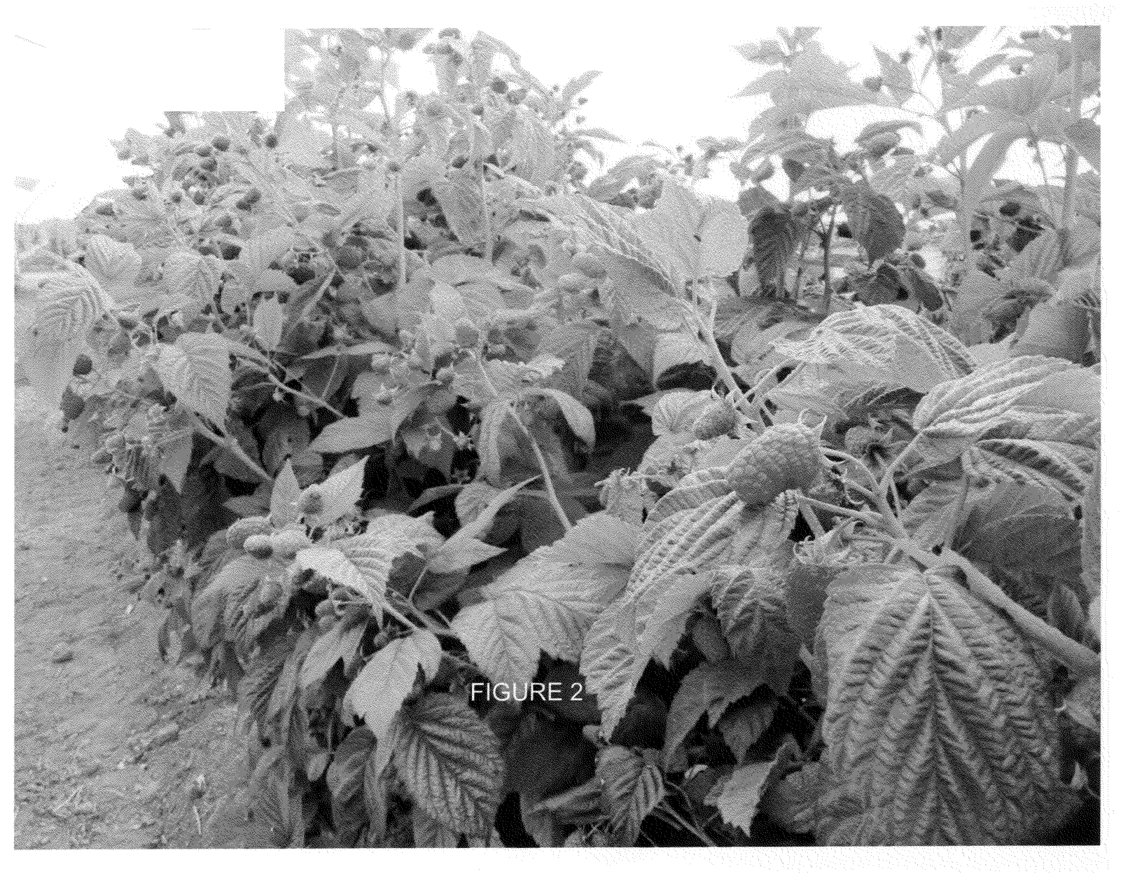

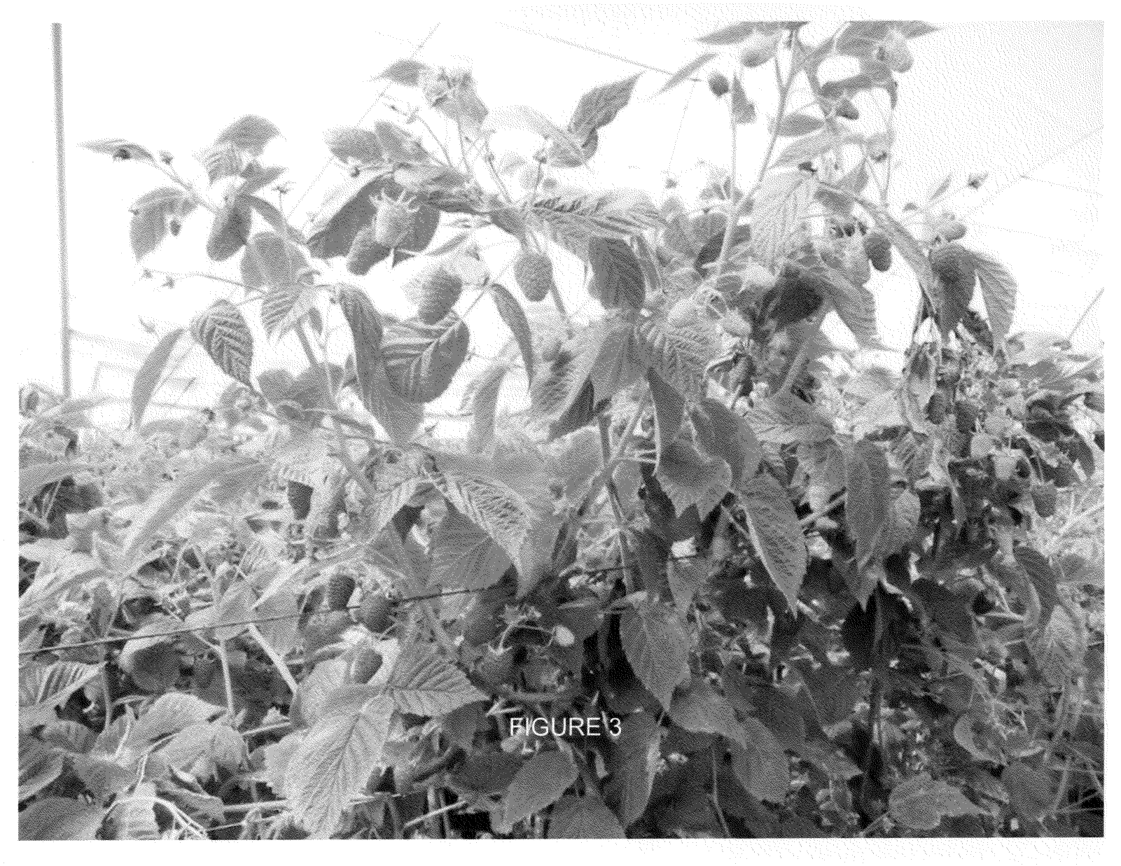

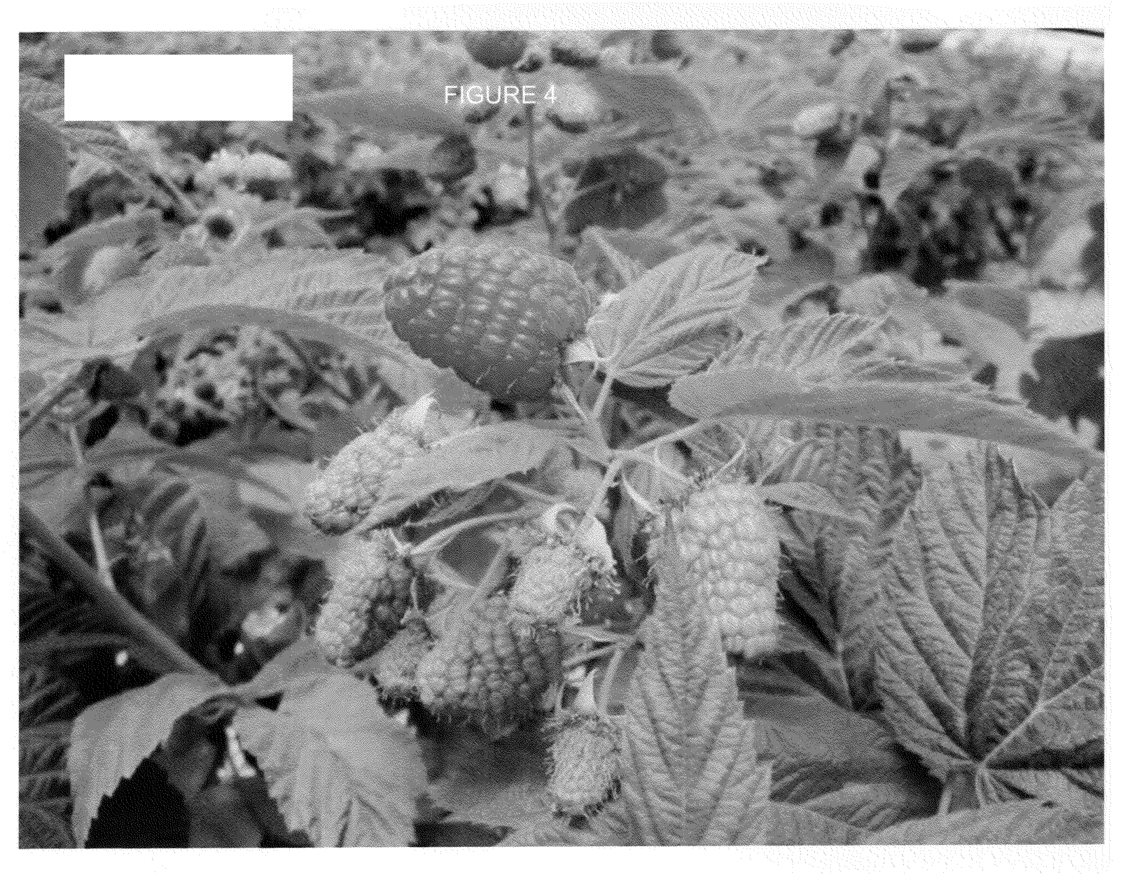

### DETAILED DESCRIPTION

Note: statements of characteristics herein represent exemplary observations of the cultivar herein and will vary depending on time of year, location, annual weather, etc.
Cultivar name: ‘Pacific Gema’
Classification:
   Family.—Rosaceae.
   Botanical name.—Rubus idaeus.
   Common name.—Raspberry.
Parentage:
   Female parent (please provide name or code).—‘Pacific Deluxe’.
   Male parent (please provide name or code flower).—‘414a’. ‘Pacific Gema’ was first identified in a field with other seedlings in May 2010 at Watsonville, Calif. USA. The field had been planted in 2009 among other seedlings generated from hand-pollinated crosses performed in 2008. ‘Pacific Gema’ was first propagated asexually by crown division in August 2010 in Watsonville, Calif. USA. The crown on the original plant was dug and parted into basal cane pieces (approximately 15 cm long) with root attached and replanted into a selection plot elsewhere on the farm, resulting in a 3-fold increase. In September 2011, two actively growing primocanes were dug (with root attached) and shipped to Lafayette, Oreg. USA, where vegetative material was explanted and established in vitro for micropropagation.
Growing location for the observations herein: Watsonville, Calif. USA.
Time of year (season): Summer.
Age of plants used for this discussion: Crown age of about 4.5 years and a cane age of about 8 months.
Age of plants used for the photographs in the figures: Crown age of about 4.5 years and a cane age of about 8 months.
Type of greenhouse covering or growing structure, or field: High tunnel over field.
Light: Natural.
References to color refer to the R.H.S. — Fifth Edition. Observations for floricanes herein were made in June 2013. Observations for primocanes herein were made in August 2013.
Plant:
   Form/shape.—Vase.
   Growth habit.—Erect.
   Height.—1.2 m as measured from base.
   Spread.—0.6 m as measured from terminal leaflet tip to terminal leaflet tip.
   Propagation methods.—stolons, tissue culture, division, tip cuttings: Division.
   Time to initiate and develop roots.—24 days.
   Root description.—Fibrous.
Primocanes:
   Diameter.—Base: 1.15 cm|Middle: 1.0 cm|Tip: 0.3 cm.
   Length.—86.5 inches.
   Number of nodes.—40-43.
   Internode length.—Base: 2.25-4 in|Middle: 1.75-4 in|Tip: 1.5-1.75 in.
   Number of canes/hill.—5-7.
   Cane color.—undertone=RHS 145B overtone=RHS 186A.
   Spines (present or absent).—present.
   Density.—Base: 5-7/cm²|Middle: 2-3/cm²|Tip: 1/cm².
   Shape.—Acute.
   Length.—0.1 cm.
   Width.—0.01 cm.
   Apex descriptor.—Acute.
   Color.—RHS 186B.
   Bud shape.—Acute.
   Length.—0.55 cm.
   Diameter (base).—0.3 cm.
   Diameter (tip).—0.1 cm.
   Color.—RHS 166A.
Floricanes:
   Diameter.—Base: 1.5 cm|Middle: 1.2 cm|Tip: 1.1 cm.
   Length.—1.1-1.2 m.
   Number of nodes.—13-16 Internode Length: 11.4 cm|17.1 cm|13.9 in.
   Cane color.—Lower Cane: RHS 199D Upper Cane: RHS 165 B.
   Spines (present or absent).—Present Density: Base: 5-7/cm²|Middle: 2-3/cm²|Tip: 1/cm² Shape: Acute Length: 0.1 cm Width: 0.1 cm Apex descriptor: Acute Color: RHS 186 B.
   Bud shape.—Acute Length: 0.4 cm Diameter (base): 0.25 cm Diameter (tip): 0.1 cm Color: RHS 166 A.
Leaves:
   Complete leaf.—Length: 12.1-18.4 cm Width: 7.6-11.4 cm Number of leaflets: 3-5.
   Terminal leaflet.—Size Length (cm): 12.1 cm Width (cm): 10.2 cm Length/Width ratio: 1.2 Shape of apex: Acuminate Shape of Base: Cordate Margin: Doubly Serrate Texture: Moderate Interveinal Puckering Number of serrations/leaf: 98-129 Shape of serrations: Flexuous — Flexuous Color: Upper Surface: RHS 136 A Lower Surface: RHS 136 D Venation pattern: Palmate Venation Color: Upper surface: RHS 144 A Lower surface: RHS 145 C Leaf pubescence density: None Color of leaf pubescence: N/A Shape of leaf in cross-section: Simple Cordate Number of leaflets/leaf: Primocane: 3 Floricane: 3-5 Interveinal blistering: Moderate Glossiness: Low.
   Primocane leaves.—Petiole length: 6.0 cm Petiole diameter: 0.2 cm Petiole Color: Upper: RHS 143C Lower: RHS N144C Rachis length: 3.3 cm Stipule length: 0.7 cm Stipules per leaf: 2 Stipule Width: 0.1 cm Stipule Color: RHS N144D Color: Upper Surface: RHS 136 A Lower Surface: RHS 136D.
   Terminal leaflet.—Length: 12.1 cm Width: 8.9 cm Rachis length: 3.3 cm.
   Distal lateral leaflet.—Not Present Length: N/A Width: N/A Petiolule length: N/A.
   Basal lateral leaflet.—Length: 9.5 cm Width: 6.4 cm Petiolule length: 0.1 cm.
   Floricane leaves.—Petiole length: 5.4 cm Stipule length: 0.7 cm Stipules per leaf: 2 Stipule Width: 0.1 cm Stipule Color: RHS N144D Color Upper surface: RHS 137A Lower surface: RHS 191B.
   Terminal leaflet.—Length: 9.5 cm Width: 7.0 in Rachis length: 1.6 cm Distal lateral leaflet: Not Present Length: N/A Width: N/A.
   Petiolule.—Length: N/A Diameter: N/A Color: N/A.
   Basal lateral leaflet.—Length: 6.4 cm Width: 4.4 cm.
   Petiolule.—Length: 0.1 cm Diameter: 0.1 cm Color: Upper: RHS 143C Lower: N144C.
Flowers:
   Time of flowering (50% of plants at first flower).—June 20 on primocanes.
   Size.—Length: 0.6 cm Diameter: 0.9 cm.
   Fragrance.—None.
   Peduncle.—Length: 0.6 cm Diameter: 0.05 cm Color: RHS 138B Pubescence: Present Texture: Smooth.
   Perianth.—Flowering trusses shape: Truncate.
   Petals.—Color (upper and lower): RHS 155C Number per flower: 5 Shape: Oblanceolate Length: 0.6 cm Width: 0.2 cm Apex descriptor: Obtuse Base Descriptor: Truncate Margin descriptor: Smooth with some undulations.
   Sepals.—Quantity: 5 Length: 1.1 cm Width: Base: 0.4 cm|Mid: 0.2 cm|Tip: 0.01 cm Color: RHS 139C Apex descriptor: Acute Margin descriptor: Smooth.
   Pedicel.—Color: RHS 144A Length: 2.8 cm Diameter: 0.1 cm.
Reproductive organs: Self-fertile: yes.
   Male.—Stamen Number: 98 Filament Length: 0.2 cm Diameter: 0.01 cm Color: RHS 157C Anther Length: 0.07 cm Diameter: 0.05 cm Color: RHS 162D Pollen Color: RHS 162D Amount: (none, sparse, moderate, heavy) Heavy.
   Female.—Style Length: 0.1 cm Diameter: 0.01 cm Color: RHS 157D Stigma Length: 0.01 cm Diameter: 0.01 cm Color: RHS 157D.
Fruit:
   Predominant shape.—Conical.
   Weight (g).—4.5 g.
   Length.—2.5 cm.
   Width.—1.5 cm.
   Length/width ratio.—1.7.
   Receptacle.—Length: 1.8 cm Diameter: Base: 0.6 cm|Mid: 0.3 cm|Tip: 0.05 cm Color: RHS 9D.
   Drupelet.—Length: 0.4 cm Diameter: 0.2 cm Number: 103 Weight: 0.2 g.
   Fruit color.—External: RHS 46A Internal: RHS 185B.
   Firmness of skin.—Moderately firm.
   Firmness of flesh.—Moderately firm.
   Hollow center.—Present.
   Number of fruit per node.—2-4.
   Time of ripening (50% of plants with first fruit).—July 28 on primocanes.
   Time of fruiting.—Late spring on floricanes, late summer and early autumn on primocanes.
   Type of bearing.—Remontant.
   Fruit yield.—24,244 lb/a/cycle.
   Average brix°.—9.44.
   Typical market use.—Fresh.
   Keeping quality.—Excellent.
   Shipping quality.—Very good.
Pest and disease resistance: Plants of ‘Pacific Gema’ have exhibited high field tolerance to Phytophthora rubi, Phragmidium rubi-idaei, and resistance to Raspberry Bushy Dwarf Virus (RBDV).

What is claimed is:

1. A new and distinct cultivar of Raspberry plant named ‘Pacific Gema’ as described and shown herein.
